(19) United States
(12) Patent Application Publication
(10) Pub. No.: US 2026/0315832 A1
(43) Pub. Date: Oct. 15, 2026

(54) DEVICE AND METHOD FOR GENERATING BUBBLES BASED ON MICROPARTICLES

(71) Applicant: ALPINION MEDICAL SYSTEMS CO., LTD., Seoul (KR)

(72) Inventors: Jeongmin Heo, Seoul (KR); Seongho KIM, Seoul (KR); Hyeonpeel CHOI, Seoul (KR)

(21) Appl. No.: 19/484,350

(22) PCT Filed: May 21, 2024
(86) PCT No.: PCT/KR2024/006860

§ 371 Date: Nov. 13, 2025

(30) Foreign Application Priority Data
May 25, 2023 (KR) 10-2023-0067417

(51) Int. Cl. A61N 7/00

(52) U.S. Cl. CPC A61N 7/00; A61N 2007/0039; A61N 2007/0073; A61N 2007/0078

(57) ABSTRACT

Provided is a device for generating bubbles based on microparticles. The device for generating bubbles based on microparticles may include a first ultrasound output unit which outputs a first ultrasound signal for moving microparticles in a blood vessel to a predetermined site, and a second ultrasound output unit which outputs a second ultrasound signal so as to generate bubbles based on the microparticles.

### TECHNICAL FIELD

The present disclosure relates to a device and method for generating bubbles based on microparticles.

### BACKGROUND ART

High-Intensity Focused Ultrasound (HIFU) may be used to treat biological tissues such as cancer, tumors, and lesions. Typically, the HIFU treatment method involves focusing high-intensity ultrasound on a single location and using the heat generated by the focused high-intensity ultrasound to ablate the biological tissues. While this HIFU treatment method has the advantage of eliminating the need for surgical incisions, the HIFU treatment method has the disadvantage of damaging healthy biological tissues due to the heat generated by the high-intensity ultrasound.

Meanwhile, a histotripsy technology is a non-thermal high-intensity focused ultrasound technology, and is a method of treating biological tissues by mechanically homogenizing lesions and tissues in a target area by creating, expanding, and collapsing cavitation bubbles in the target area through control of the pressure and frequency of high-intensity focused ultrasound. According to this histotripsy, it is possible to reduce the risk of damage to surrounding areas outside the target area compared to the conventional HIFU.

Recent research by various domestic and international institutions and companies has led to the development of various histotripsy technologies, including methods that generate cavitation bubbles using strong negative pressure and methods that use strong positive pressure to raise a tissue temperature to 100° C. and then generate boiling bubbles.

However, the currently developed methods require high-performance/large-scale driving equipment because they must output the ultrasound signal with a pressure stronger than the pressure (cavitation threshold) at which bubbles are generated to address the acoustic attenuation problem where the pressure is attenuated before reaching the target area.

The technology underlying the present disclosure is disclosed in U.S. Patent Publication Ser. No. 12/358,549.

### DISCLOSURE

### Technical Problem

The present disclosure aims to address the problems of the related art described above, and an object thereof is to provide a device and method for generating bubbles based on microparticles capable of generating bubbles at a pressure lower than the cavitation threshold by inducing scattering in a sound field containing captured microparticles, thereby generating bubbles.

However, objects to be achieved by the embodiments of the present disclosure are not limited to the objects described above, and other objects may exist.

### Technical Solution

In order to achieve the above-described object, according to one embodiment of the present disclosure, there is provided a device for generating bubbles based on microparticles, the device including: a first ultrasound output unit that outputs a first ultrasound signal for moving microparticles in a blood vessel to a predetermined site; and a second ultrasound output unit that outputs a second ultrasound signal so as to generate bubbles based on the microparticles.

According to one embodiment of the present disclosure, the second ultrasound output unit may output the second ultrasound signal toward the microparticles or the surroundings of the microparticles.

According to one embodiment of the present disclosure, the second ultrasound output unit may generate the bubbles based on the second ultrasound signal reflected and scattered from the microparticles or the surroundings of the microparticles.

According to one embodiment of the present disclosure, the bubbles may be cavitation bubbles for fragmenting a tissue approaching within a predetermined distance from the predetermined site.

According to one embodiment of the present disclosure, the first ultrasound output unit may generate a sound field that captures the microparticles using the first ultrasound signal.

According to one embodiment of the present disclosure, the second ultrasound output unit may output the second ultrasound signal toward the sound field and generate the bubbles based on the second ultrasound signal reflected and scattered from a microparticle cluster composed of the microparticles captured within the sound field.

According to one embodiment of the present disclosure, the sound field may have a ring-shaped pressure distribution in which no pressure exists in a central region and pressure exists in the peripheral region.

According to one embodiment of the present disclosure, the first ultrasound output unit may determine a size of a ring according to a pressure distribution of the sound field by adjusting a topological charge of the first ultrasound signal, and determine a size of an ultrasound focal area according to the size of the ring.

According to one embodiment of the present disclosure, the first ultrasound output unit may output a plurality of channel signals having different phase differences through a plurality of ultrasound output channels.

According to one embodiment of the present disclosure, the second ultrasound signal may have a pressure amplitude less than a cavitation threshold.

According to one embodiment of the present disclosure, the second ultrasound output unit may focus the second ultrasound signal on the sound field, induce scattering of the second ultrasound signal from the microparticle cluster within the sound field, and generate a scattered sound field having a pressure amplitude equal to or more than the cavitation threshold from the scattering of the second ultrasound signal.

According to one embodiment of the present disclosure, the second ultrasound output unit may use the second ultrasound signal to induce collapse of the bubbles and the microparticle cluster generated by the scattered sound field, thereby fragmenting at least a portion of the tissue.

According to one embodiment of the present disclosure, the cavitation threshold may be a minimum pressure at which the bubbles can be generated when only the second ultrasound signal is used.

In order to achieve the above-described object, according to one embodiment of the present disclosure, there is provided a method for generating bubbles based on microparticles, the method including: outputting a first ultrasound signal to move microparticles in a blood vessel to a predetermined site; and outputting a second ultrasound signal to generate bubbles based on the microparticles.

According to one embodiment of the present disclosure, the outputting of the second ultrasound signal may include outputting the second ultrasound signal toward the microparticles or the surroundings of the microparticles.

According to one embodiment of the present disclosure, the outputting of the second ultrasound signal may include generating the bubbles based on the second ultrasound signal reflected and scattered from the microparticles or the surroundings of the microparticles.

According to one embodiment of the present disclosure, the outputting of the first ultrasound signal may include generating a sound field that captures the microparticles using the first ultrasound signal.

According to one embodiment of the present disclosure, the outputting of the second ultrasound signal may include outputting the second ultrasound signal toward the sound field and generating the bubbles based on the second ultrasound signal reflected and scattered from a microparticle cluster composed of the microparticles captured within the sound field.

According to one embodiment of the present disclosure, the outputting of the first ultrasound signal may include determining a size of a ring according to a pressure distribution of the sound field by adjusting a topological charge of the first ultrasound signal, and determining a size of an ultrasound focal area according to the size of the ring.

According to one embodiment of the present disclosure, the outputting of the first ultrasound signal may include outputting a plurality of channel signals having different phase differences through a plurality of ultrasound output channels.

According to one embodiment of the present disclosure, the outputting of the second ultrasound signal may include focusing the second ultrasound signal on the sound field, inducing scattering of the second ultrasound signal from the microparticle cluster within the sound field, and generating a scattered sound field having a pressure amplitude equal to or more than the cavitation threshold from the scattering of the second ultrasound signal.

According to one embodiment of the present disclosure, the outputting of the second ultrasound signal may include using the second ultrasound signal to induce collapse of the bubbles and the microparticle cluster generated by the scattered sound field, thereby fragmenting at least a portion of the tissue.

The above-described technical solutions are merely exemplary and should not be construed as limiting the present disclosure. In addition to the exemplary embodiments described above, additional embodiments may be included in the drawings and detailed description of the invention.

### Advantageous Effects

According to the technical solutions of the present disclosure described above, the bubbles can be generated at a lower pressure than the cavitation threshold by inducing scattering in the sound field that captures microparticles, thereby generating bubbles.

However, effects obtained in the present disclosure are not limited to the effects described above, and other effects may exist.

### DESCRIPTION OF DRAWINGS

FIG. 1 is a schematic block diagram of a device for generating bubbles based on microparticles according to one embodiment of the present disclosure.

FIG. 2 is a schematic diagram of a transducer of the device for generating bubbles based on microparticles according to one embodiment of the present disclosure.

FIG. 3 is a diagram visually representing waveforms of a first ultrasound signal and a second ultrasound signal output from the device for generating bubbles based on microparticles according to one embodiment of the present disclosure.

FIG. 4 is a diagram visually representing a pressure distribution of the first ultrasound signal output from the device for generating bubbles based on microparticles according to one embodiment of the present disclosure.

FIG. 5 is a diagram visually representing the pressure distribution and phase distribution of the first ultrasound signal output from the device for generating bubbles based on microparticles according to one embodiment of the present disclosure.

FIG. 6 is a diagram illustrating a process of capturing microparticles using a sound field generated by the device for generating bubbles based on microparticles according to one embodiment of the present disclosure.

FIG. 7 is a diagram illustrating a process of generating bubbles using the device for generating bubbles based on microparticles according to one embodiment of the present disclosure.

FIG. 8 is a diagram illustrating a pressure amplitude in tissue when bubbles are generated using a sound field generated from the device for generating bubbles based on microparticles according to one embodiment of the present disclosure, and when bubbles are generated without generating the sound field.

FIG. 9 is a flowchart illustrating a method for generating bubbles based on microparticles according to one embodiment of the present disclosure.

### BEST MODE

Below, with reference to the attached drawings, embodiments of the present disclosure are described in detail so that those skilled in the art can easily practice the present disclosure. However, the present disclosure may be implemented in various different forms and is not limited to the embodiments described herein. Furthermore, for clarity in the drawings, portions irrelevant to the description have been omitted, and similar parts are designated with similar reference numerals throughout the specification.

Throughout this specification, when a part is referred to as being “connected” to another part, this includes not only “directly connected” but also “electrically connected” or “indirectly connected” with another element intervening.

Throughout this specification, when a member is referred to as being “on”, “above”, “at an upper end of”, “below”, “under”, or “at a lower end of” another member, this includes not only cases where the member is in contact with the other member, but also cases where another member exists between the two members.

Throughout this specification, when a part is said to “include” a component, this does not mean that other components are excluded, but rather that other components may be included, unless otherwise specifically stated.

The present disclosure relates to a device and method for generating bubbles based on microparticles.

Generally, high-intensity ultrasound may refer to ultrasound that is approximately 100,000 times stronger than diagnostic ultrasound. A high-intensity focused ultrasound (HIFU) treatment method is a treatment method that burns and removes biological tissue in a specific area by concentrating high-intensity ultrasound on one location (specific area) and generating high heat of 65° C. to 100° C. in the specific area. This HIFU treatment method has the advantage of eliminating the need for surgical incisions, but may have the disadvantage of damaging healthy biological tissue due to the heat generated by the high-intensity ultrasound.

Meanwhile, the histotripsy method described in the present disclosure is a high-intensity focused ultrasound treatment method using a non-thermal method, and may refer to a method of treating biological tissues by controlling the pressure and frequency of high-intensity focused ultrasound to generate, expand, and collapse cavitation bubbles in a target area, mechanically homogenizing lesions and tissues in the target area. The histotripsy may reduce the risk of damage to surrounding areas outside the target area compared to conventional HIFU treatment methods.

Hereinafter, for convenience of explanation, a “device 100 for generating bubbles based on microparticles” according to one embodiment of the present disclosure will be referred to as a “bubble generation device 100”.

The bubble generation device 100 according to one embodiment of the present disclosure may include an ultrasound probe and a computer unit that controls the ultrasound probe. Specifically, the bubble generation device 100 may include an ultrasound probe including at least one transducer that outputs an ultrasound signal, and a computing device programmed with instructions for controlling the ultrasound signal output from the at least one transducer. A first ultrasound output unit 110 and a second ultrasound output unit 120 of the bubble generation device 100, which will be described below, may be interpreted as either a transducer that outputs the first ultrasound signal and the second ultrasound signal, or a control unit that controls the ultrasound signal output of the transducer, respectively, or as including a transducer and a control unit.

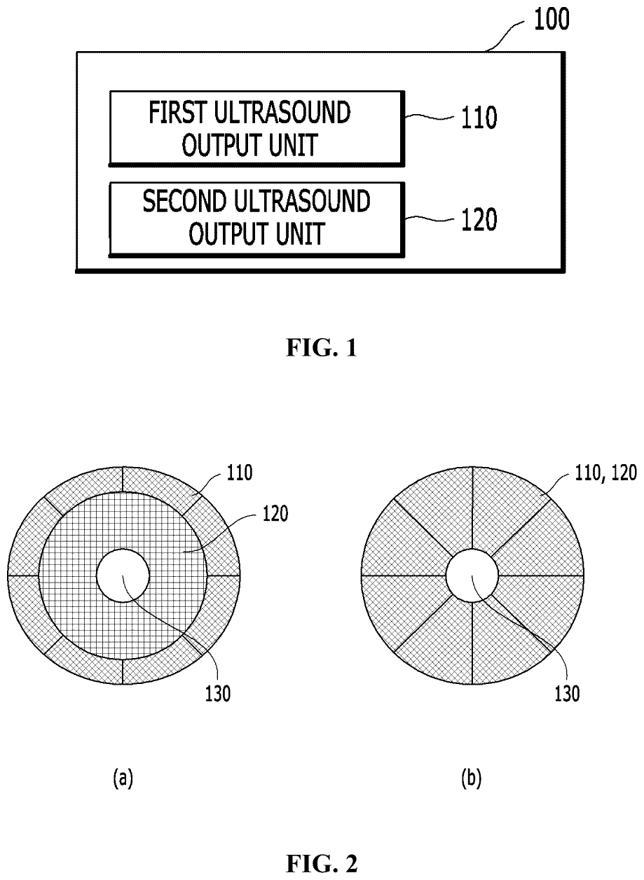

FIG. 1 is a schematic block diagram of the device for generating bubbles based on microparticles according to one embodiment of the present disclosure.

Referring to FIG. 1, the bubble generation device 100 may include the first ultrasound output unit 110 and the second ultrasound output unit 120.

According to one embodiment of the present disclosure, the first ultrasound output unit 110 may output a first ultrasound signal that moves microparticles 2 within a blood vessel to a predetermined site. In this case, the microparticles 2 may be an ultrasound contrast agent (microbubble injection) injected into the blood vessel of a subject (for example, a patient, or the like) through an injection, but is not limited thereto.

In addition, the first ultrasound signal is a signal for manipulating microparticles 2 called acoustic tweezers, and may be a signal having a pressure of 100 kPa or more and 10 MPa or less (since it is a symmetrical waveform, the negative and positive pressures are the same), a pulse repetition frequency (PRF) of 1 Hz or more and 500 Hz or less, and a frequency of 20 kHz or more and 50 MHz or less, but is not limited thereto.

In other words, the first ultrasound output unit 110 may move microparticles 2 injected into a blood vessel and flowing within the blood vessel to a predetermined site using the first ultrasound signal, which is an acoustic tweezers signal. Specifically, the first ultrasound output unit 110 may generate a sound field 1 that captures the microparticles 2 using the first ultrasound signal, and move the captured microparticles 2 to a predetermined site using the generated sound field 1.

According to one embodiment of the present disclosure, the second ultrasound output unit 120 may output a second ultrasound signal so that bubbles are generated based on the microparticles 2.

In this case, the second ultrasound signal is a high-intensity focused ultrasound (HIFU) signal for generating the bubbles 5 from the microparticles 2 captured by the first ultrasound signal or moved to a predetermined site, and the second ultrasound signal may have a positive pressure (P+) of 1 MPa or more and 100 MPa or less or a negative pressure (P−) of −25 MPa or less, a pulse repetition frequency (PRF) of 1 Hz or more and 500 Hz or less, or a frequency of 100 kHz or more and 50 MHz or less, but is not limited thereto.

The second ultrasound output unit 120 may output the second ultrasound signal toward the microparticles 2 or the surroundings of the microparticles 2, and generate the bubbles 5 based on the second ultrasound signal reflected and scattered from the microparticles 2 or the surroundings of the microparticles 2.

Specifically, the second ultrasound output unit 120 may focus the second ultrasound signal on the sound field 1 moved to a predetermined site by the first ultrasound signal or on the microparticles 2 captured in the sound field 1, thereby amplifying the pressure of the predetermined site to a pressure (cavitation threshold: minimum pressure at which cavitation bubbles can be generated) at which cavitation bubbles can be generated to generate scattering from the microparticles 2 or the surroundings of the microparticles 2, and generate bubbles 5 through the generated scattering. In addition, the second ultrasound output unit 120 may induce the collapse of the generated bubbles 5, thereby fragmenting or removing a tissue site due to the collapse of the bubbles 5. Here, the tissue may refer to cancer and tumors, but is not limited thereto.

Here, the predetermined site may refer to the location of the tissue site to be fragmented or removed, and the bubbles 5 may be cavitation bubbles for fragmenting or removing the tissue site. In other words, the bubbles 5 may be the cavitation bubbles for fragmenting tissue approaching within a predetermined distance from the predetermined site.

According to one embodiment of the present disclosure, the first ultrasound output unit 110 and the second ultrasound output unit 120 may be provided in a single array or multi-array form depending on the structure of the transducer, and may include the plurality of (N) beamforming sources to enable both forms. In addition, the second ultrasound output unit 120 may include a high-voltage source, as the second ultrasound output unit 120 should output a high-intensity ultrasound signal in a short period of time to fragment or remove tissue.

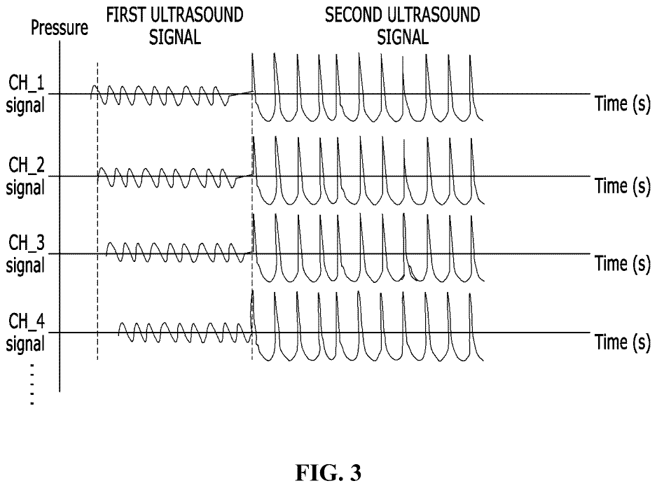

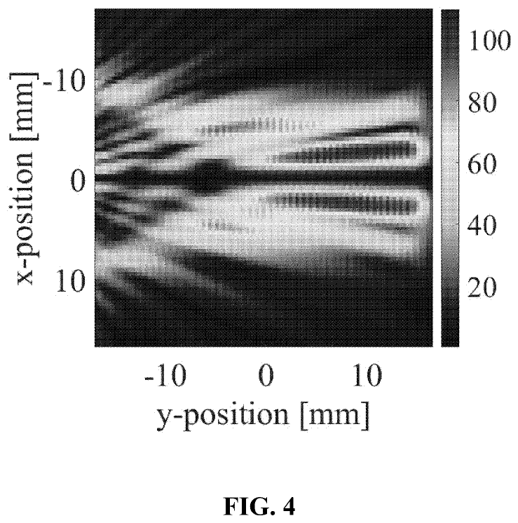

Here, the first ultrasound output unit 110 includes a plurality of ultrasound output channels, and should control the plurality of ultrasound output channels so that the signals after the first channel signal are each delayed (that is, have different phase differences) based on the first channel signal, and the second ultrasound output unit 120 should also control or delay the plurality of ultrasound output channels simultaneously according to the first channel signal of the first ultrasound output unit 110. Accordingly, the beamforming source of the first ultrasound output unit 110 and the beamforming source of the second ultrasound output unit 120 may be configured to be divided into each group, and a plurality of multiplexers (MUXs) may be configured according to each group. In other words, the first ultrasound signal and the second ultrasound signal may be controlled, respectively, through the plurality of MUXs. The configuration related to the ultrasound output channels and the delay (phase difference) of the plurality of channel signals of the first ultrasound output unit 110 and the second ultrasound output unit 120 will be described in detail later with reference to FIGS. 3 and 4.

FIG. 2 is a schematic diagram of the transducer of the device for generating bubbles based on microparticles according to one embodiment of the present disclosure.

(a) of FIG. 2 illustrates a first embodiment of the transducer arrangement of the bubble generation device 100, and (b) of FIG. 2 illustrates a second embodiment of the transducer arrangement of the bubble generation device 100.

Referring to FIG. 2, the transducer of the bubble generation device 100 may be formed in a circular shape. However, the present disclosure is not limited thereto, and the transducer may be provided in various shapes, such as an oval or a square shape. More specifically, referring to (a) of FIG. 2, the first ultrasound output unit 110, the second ultrasound output unit 120, and an imaging ultrasound output unit 130 may be configured in a concentric circle shape, and even if not circular, may be configured in an oval, square, or other shape that shares a center.

In addition, as described above, the first ultrasound output unit 110 and the second ultrasound output unit 120 may be interpreted as ultrasound transducers included in an ultrasound probe. That is, in describing FIG. 2, the first ultrasound output unit 110 may mean a first transducer that outputs a first ultrasound signal, and the second ultrasound output unit 120 may mean a second transducer that outputs a second ultrasound signal.

Referring to (a) of FIG. 2, the first ultrasound output unit 110 may be positioned at the outermost end of the output surface of the ultrasound probe. In this case, the first ultrasound output unit 110 may include a plurality of ultrasound output channels and output a plurality of channel signals having different phase differences through the plurality of ultrasound output channels. For example, referring to FIG. 2, the first ultrasound output unit 110 may include eight ultrasound output channels, but the present disclosure is not limited thereto.

Referring to (a) of FIG. 2, the second ultrasound output unit 120 may be positioned inside the first ultrasound output unit 110 on the output surface of the ultrasound probe.

Referring to (a) of FIG. 2, the bubble generation device 100 may further include an imaging ultrasound output unit 130. According to one embodiment of the present disclosure, the imaging ultrasound output unit 130 may transmit an imaging ultrasound signal for performing a monitoring function and receive a reflected ultrasound signal. Here, the monitoring function may include, for example, detecting tissue requiring treatment, monitoring the tissues, microparticles 2, the sound field 1, and generated bubbles 5, or monitoring tissue during or after treatment with the bubbles 5, but is not limited thereto. In addition, the imaging ultrasound output unit 130 may be an additional component that is not an essential component of the present disclosure and may not be provided in one embodiment of the present disclosure.

As described above with reference to (a) of FIG. 2, the bubble generation device 100 may include the first transducer corresponding to the first ultrasound output unit 110, the second transducer corresponding to the second ultrasound output unit 120, and the imaging transducer corresponding to the imaging ultrasound output unit 130. Each transducer may be an individual transducer, but is not limited thereto, and may be integrated as a single transducer.

For example, the first ultrasound output unit 110 and the second ultrasound output unit 120 may be provided as a single transducer, and may output the first ultrasound signal and the second ultrasound signal through the plurality of channels or by manipulating the ultrasound signal.

Referring to (b) of FIG. 2, the first ultrasound output unit 110 and the second ultrasound output unit 120 may be provided as a single transducer. In this case, the bubble generation device 100 may include a plurality of ultrasound output channels, and output a first ultrasound signal including a plurality of channel signals with different phase differences through the plurality of ultrasound output channels, and the bubble generation device 10 may output the second ultrasound signal by adjusting the phase, pressure amplitude, PRF, and frequency of each ultrasound output channel in the first ultrasound signal.

FIG. 3 is a diagram visually representing the waveforms of the first ultrasound signal and the second ultrasound signal output by the device for generating bubbles based on microparticles according to one embodiment of the present disclosure.

Referring to FIG. 3, it can be confirmed that the first ultrasound signal has a different phase difference depending on each ultrasound output channel, but the second ultrasound signal operates with the same phase in all ultrasound output channels. Accordingly, the second ultrasound output unit 120 that outputs the second ultrasound signal may be the unit in which the second transducer is not divided into the plurality of ultrasound output channels, as in (a) of FIG. 2, or may be the unit in which the second transducer is divided into the plurality of ultrasound output channels, as in (b) of FIG. 2. In the case of (b) of FIG. 2, the second ultrasound output unit 120 may output the second ultrasound signal through the plurality of divided ultrasound output channels, and may output the plurality of channel signals output from the plurality of ultrasound output channels all with the same phase, thereby outputting the second ultrasound signal.

Hereinafter, the process of generating bubbles by the first ultrasound signal and the second ultrasound signal output from the bubble generation device 100 according to one embodiment of the present disclosure will be described in more detail.

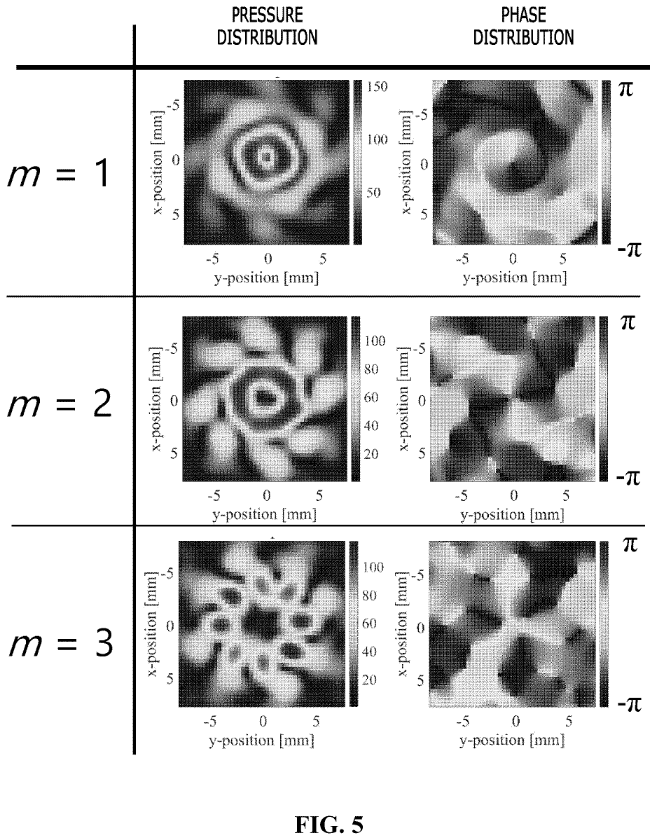

FIG. 4 is a diagram visually representing the pressure distribution of the first ultrasound signal output from the device for generating bubbles based on microparticles according to one embodiment of the present disclosure. Additionally, FIG. 5 is a diagram visually representing the pressure distribution and phase distribution of the first ultrasound signal output from the device for generating bubbles based on microparticles according to one embodiment of the present disclosure.

Referring to one embodiment of the present disclosure, the first ultrasound output unit 110 may induce destructive interference between signals (waves) output from each channel by outputting the plurality of channel signals having different phase differences as the first ultrasound signal through the plurality of ultrasound output channels as described above. Due to the interference between these signals, a sound field 1 is generated with a ring-shaped pressure distribution in which there is no pressure in the central region and there is a pressure in the peripheral regions (refer to the pressure distributions in FIGS. 4 and 5).

In other words, referring to FIGS. 4 and 5, the first ultrasound output unit 110 may output the plurality of channel signals with different phase differences, induce destructive interference between the signals, thereby generating a ring-shaped sound field 1 with a central pressure of zero.

In this regard, each of the plurality of channel signals may be determined using Expression 1 below.

$${A}_{n} = {P}_{0} sin ( 2 π ft + n \frac{2 π m}{N} ) \qquad [ Expression 1 ]$$

Here, N represents the total number of channels, n represents the channel order (channel number), P₀ represents the pressure amplitude of each channel signal, f represents the ultrasound frequency, t represents time, and m represents the topological charge.

Here, the topological charge refers to a physical quantity that takes only one of the given discrete values due to the topological properties of the mathematical model of a theory explaining a physical phenomenon, and is also called a topological quantum number.

According to one embodiment of the present disclosure, the first ultrasound output unit 110 may control the size of the ring area of the sound field 1 by adjusting the topological charge m of the first ultrasound signal. Referring to the pressure distribution of FIG. 5, it can be confirmed that the size of the ring area with the central pressure of 0 according to the pressure distribution of the first ultrasound signal is larger when the topological charge m is 3 than when the topological charge is 1. In this case, the size of the ring area may indicate the size of the sound field 1. Furthermore, since the bubbles 5 are generated by the second ultrasound signal scattered from the sound field 1, and tissue fragmentation occurs through the collapse of the generated bubbles 5, the size of the sound field 1 may refer to the size of the focal area where the ultrasound is focused for treatment (removal).

In other words, the first ultrasound output unit 110 may adjust the topological charge m of the plurality of channel signals of the first ultrasound signal to determine the size of the ring according to the pressure distribution of the sound field 1, and determine the size of the ultrasound focal area according to the size of the ring.

Referring to the phase distribution in FIG. 5, although there are slight differences depending on the topological charge m, it can be confirmed that the phase distribution of the first ultrasound signal rotates around the central area, regardless of the value of the topological charge m. In other words, the sound field 1 generated by the first ultrasound signal may rotate from π(180°) to −π(−180°). The first ultrasound output unit 110 may utilize this rotation of the sound field 1 to move the sound field 1 containing the captured microparticles 2 to the tissue site (a predetermined site) requiring treatment.

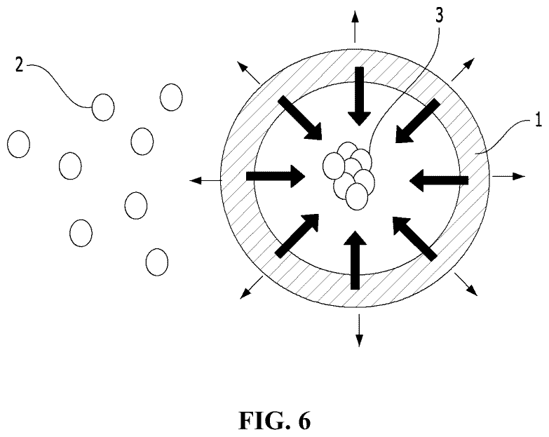

FIG. 6 is a diagram illustrating a process of capturing the microparticles using the sound field generated by the device for generating bubbles based on microparticles according to one embodiment of the present disclosure.

Referring to FIG. 6, the central region of the sound field 1 generated by the first ultrasound signal may have a pressure of 0, and the microparticles 2 outside the sound field 1 may be attracted to the central region of the sound field 1 and clustered due to the pressure difference between the central region, the surrounding region, and the external region of the sound field 1. Hereinafter, a group of microparticles clustered in the central region of a sound field 1 will be referred to as a microparticle cluster 3.

According to this, the first ultrasound output unit 110 may generate the sound field 1 using the plurality of channel signals with different phase differences, and use the pressure difference between regions of the sound field 1 to cause the microparticles 2 to cluster in the central region of the sound field 1. In this case, the size of the microparticles 2 may be 3 to 6 μm, and the size of the microparticle cluster 3 generated by the clustering of microparticles 2 may reach several hundred μm.

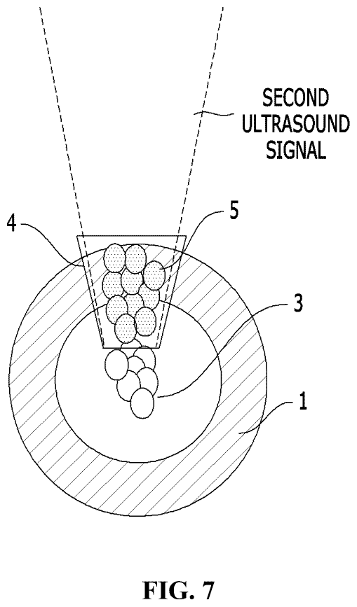

FIG. 7 is a diagram illustrating a process for generating the bubbles using the device for generating bubbles based on microparticles 2 according to one embodiment of the present disclosure.

Referring to FIG. 7, when the second ultrasound signal is applied to the sound field 1 including the microparticle cluster 3 in the central region by the second ultrasound output unit 120, the second ultrasound signal is scattered from the microparticle cluster 3, thereby generating a scattered sound field 4.

In this case, the scattered sound field 4 may have a pressure amplitude that is about twice as strong as that of the second ultrasound signal, and the bubbles 5 may be generated due to the strong pressure amplitude of this scattered sound field 4.

As described above, the second ultrasound output unit 120 may explosively increase the pressure amplitude using the scattered sound field 4. Therefore, even when a second ultrasound signal with a pressure amplitude lower than the pressure amplitude (cavitation threshold) at which bubbles 5 are generated is applied, the bubbles 5 for tissue fragmentation may be generated at a predetermined site.

That is, the second ultrasound signal output from the second ultrasound output unit 120 may have the pressure amplitude lower than the cavitation threshold. Here, the cavitation threshold generally refers to the critical pressure for the generation of cavitation bubbles, that is, the minimum pressure at which cavitation bubbles can be generated, and may be approximately −28 MPa. In other words, the cavitation bubbles can be generated when the pressure amplitude is approximately −28 MPa or lower.

The second ultrasound output unit 120 may amplify the pressure amplitude in the surrounding area of the sound field 1 and the microparticle cluster 3 by approximately twice that of the second ultrasound signal by generating the scattered sound field 4 using the sound field 1. Therefore, even when the pressure amplitude of the second ultrasound signal is approximately half the cavitation threshold, the bubbles 5 can be sufficiently generated.

For example, the second ultrasound signal may be approximately −16 MPa, and the scattered sound field 4 may be approximately −30 MPa, but the present disclosure is not limited thereto.

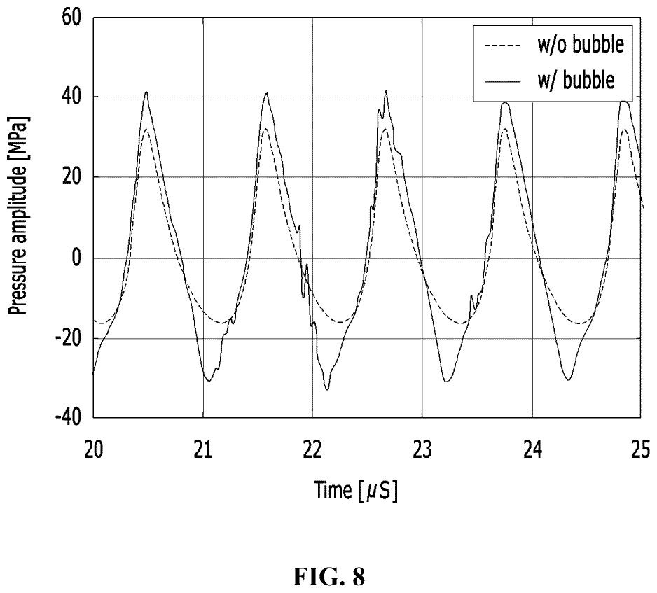

FIG. 8 is a diagram illustrating the pressure amplitude in tissue when bubbles are generated using the sound field 1 generated from the device for generating bubbles based on microparticles 2 according to one embodiment of the present disclosure, and when bubbles are generated without generating the sound field 1. Referring to FIG. 8, when the second ultrasound signal has a negative pressure of about −16 MPa, in a case where the sound field 1 and the microparticle cluster 3 exist at the predetermined site (w/bubble), the sound field 1 at the predetermined site and the pressure around the sound field 1 become about 41 MPa(P+)/−30 MPa(P−), so that bubbles 5 may be generated. Meanwhile, when the sound field 1 is not generated at the predetermined site, the microparticles are clustered together, and the microparticle cluster 3 is not generated (w/o bubble), the pressure is approximately 31 MPa(P+)/−16 MPa(P−), which is similar to the second ultrasound signal to which the pressure of the predetermined site is applied, and thus, the pressure does not reach the cavitation threshold, so that bubbles 5 are not generated. In addition, the second ultrasound output unit 120 may use the second ultrasound signal to induce the collapse of the microparticle cluster 3 within the sound field 1 and the bubbles 5 generated by the scattered sound field 4, thereby fragmenting at least a portion of the tissue.

That is, according to one embodiment of the present disclosure, the bubble generation device 100 may generate the sound field 1 and move the sound field 1 to a predetermined site, capture the microparticles 2 with the sound field 1 to generate the microparticle cluster 3, focus high-intensity ultrasound toward the sound field 1 to generate the scattered sound field 4 and bubbles 5 from the sound field 1, and induce the collapse of the sound field 1, the microparticle cluster 3, and the bubbles 5, thereby treating the tissue at the predetermined site.

According to the above, the bubble generation device 100 may generate the bubbles 5 at a pressure lower than the minimum pressure for generating the cavitation bubbles, that is, the cavitation threshold, using only high-intensity focused ultrasound by inducing scattering in the sound field 1 that captures microparticles 2 to generate the bubbles 5.

In addition, the bubble generation device 100 may significantly reduce the pressure amplitude of the ultrasound signal that should be applied to generate the bubbles 5, thereby making unnecessary the high-performance/large-scale driving equipment required to output a high-pressure ultrasound signal. Therefore, when implementing the device, the required equipment specifications may be lowered and the entire system may be miniaturized.

In addition, the bubble generation device 100 may adjust the size of the ultrasound focal area using the sound field 1, and after moving the sound field 1 to a predetermined site, induce the collapse of the microparticle cluster 3 and bubbles 5, thereby enabling tissue treatment at a predetermined site with high precision.

Below, based on the detailed description above, the operational flow of the present disclosure will be briefly described.

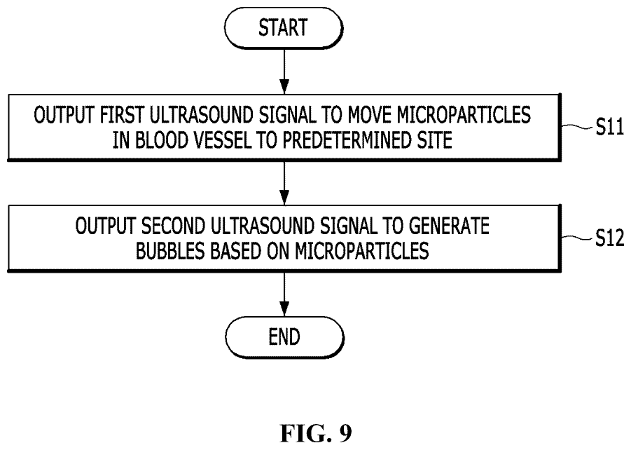

FIG. 9 is a flowchart illustrating a method for generating bubbles based on the microparticles 2 according to one embodiment of the present disclosure.

The method for generating bubbles based on the microparticles 2 illustrated in FIG. 9 may be performed by the device 100 for generating bubbles based on the microparticles 2 described above. Therefore, even when omitted below, the description of the device 100 for generating bubbles based on the microparticles 2 may be equally applied to the description of the method for generating bubbles based on the microparticles 2.

Referring to FIG. 9, in Step S11, the first ultrasound output unit 110 may output the first ultrasound signal to move the microparticles 2 in a blood vessel to the predetermined site.

Next, in Step S12, the second ultrasound output unit 120 may output the second ultrasound signal to generate the bubbles based on the microparticles 2. Specifically, in Step S12, the second ultrasound output unit 120 may output the second ultrasound signal toward the microparticles 2 or the surroundings of the microparticles 2, and generate the bubbles based on the second ultrasound signal reflected and scattered from the microparticles 2 or the surroundings of the microparticles 2. In this case, the bubbles may be the cavitation bubbles for fragmenting tissue approaching within a predetermined distance from the predetermined site.

More specifically, in Step S11, the first ultrasound output unit 110 may generate the sound field 1 that captures the microparticles 2 using the first ultrasound signal. In this case, the sound field 1 may have a ring-shaped pressure distribution in which there is no pressure in the central region and there is a pressure in the peripheral region.

In addition, in Step S11, the first ultrasound output unit 110 may adjust the topological charge of the first ultrasound signal to determine the size of the ring according to the pressure distribution of the sound field 1, and determine the size of the ultrasound focal area according to the size of the ring. In addition, in Step S11, the first ultrasound output unit 110 may output the plurality of channel signals having different phase differences through the plurality of ultrasound output channels.

Next, in Step S12, the second ultrasound output unit 120 may output the second ultrasound signal toward the sound field 1, and generate the bubbles based on the second ultrasound signal reflected and scattered from the cluster of the microparticles 2 composed of the microparticles 2 captured within the sound field 1. In this case, the second ultrasound signal may have the pressure amplitude lower than the cavitation threshold, which means the minimum pressure at which bubbles can be generated when only the second ultrasound signal is used without using the sound field 1.

In other words, in Step S12, the second ultrasound output unit 120 may focus the second ultrasound signal onto the sound field 1, induce scattering of the second ultrasound signal from a cluster of microparticles 2 within the sound field 1, generate the scattered sound field 4 having the pressure amplitude greater than a cavitation threshold from the scattering of the second ultrasound signal, and use the second ultrasound signal to induce collapse of the bubbles and the cluster of microparticles 2 generated by the scattered sound field 4, thereby fragmenting at least a portion of the tissue.

In the above description, Steps S11 and S12 may be further divided into additional steps or combined into fewer steps, depending on the implementation examples of the present disclosure. Furthermore, some steps may be omitted as needed, and the order between the steps may be changed.

The method for generating bubbles based on microparticles 2 according to one embodiment of the present disclosure may be implemented in the form of program commands that can be executed through various computer means and recorded on a computer-readable medium. The computer-readable medium may include program commands, data files, data structures, or the like, alone or in combination. The program commands recorded on the medium may be those specially designed and configured for the present disclosure or may be those known and available to those skilled in the art of computer software. Examples of the computer-readable recording medium include magnetic media such as hard disks, floppy disks, and magnetic tapes, optical media such as CD-ROMs and DVDs, magneto-optical media such as floptical disks, and hardware devices specially configured to store and execute program commands such as ROMs, RAMs, and flash memories. Examples of the program commands include not only machine language codes generated by a compiler, but also high-level language codes that can be executed by a computer using an interpreter, or the like. The hardware device described above may be configured to operate as one or more software modules to perform the operations of the present disclosure, and vice versa.

Furthermore, the method for generating bubbles based on the microparticles 2 described above may also be implemented in the form of a computer program or application stored on a recording medium and executed by a computer.

The description of the present disclosure described above is for illustrative purposes only, and those skilled in the art will understand that the present disclosure can be easily modified into other specific forms without altering the technical spirit or essential characteristics of the present disclosure. Therefore, it should be understood that the embodiments described above are illustrative in all respects and not restrictive. For example, each component described as a single component may be implemented in a distributed manner, and similarly, components described as distributed may be implemented in a combined manner.

The scope of the present disclosure is determined by the claims described below rather than the detailed description above, and all changes or modifications derived from the meaning and scope of the claims and their equivalents should be construed as being included within the scope of the present disclosure.

1. A device for generating bubbles based on microparticles, the device comprising:
a first ultrasound output unit that outputs a first ultrasound signal for moving microparticles in a blood vessel to a predetermined site; and
a second ultrasound output unit that outputs a second ultrasound signal so as to generate bubbles based on the microparticles.

2. The device of claim 1, wherein the second ultrasound output unit outputs the second ultrasound signal toward the microparticles or surroundings of the microparticles.

3. The device of claim 2, wherein the second ultrasound output unit generates the bubbles based on the second ultrasound signal reflected and scattered from the microparticles or the surroundings of the microparticles.

4. The device of claim 1, wherein the bubbles are cavitation bubbles for fragmenting a tissue approaching within a predetermined distance from the predetermined site.

5. The device of claim 4, wherein the first ultrasound output unit generates a sound field that captures the microparticles using the first ultrasound signal.

6. The device of claim 5, wherein the second ultrasound output unit outputs the second ultrasound signal toward the sound field and generates the bubbles based on the second ultrasound signal reflected and scattered from a microparticle cluster composed of the microparticles captured within the sound field.

7. The device of claim 5, wherein the sound field has a ring-shaped pressure distribution in which no pressure exists in a central region and pressure exists in a peripheral region.

8. The device of claim 7, wherein the first ultrasound output unit determines a size of a ring according to the pressure distribution of the sound field by adjusting a topological charge of the first ultrasound signal, and determines a size of an ultrasound focal area according to the size of the ring.

9. The device of claim 1, wherein the first ultrasound output unit outputs a plurality of channel signals having different phase differences through a plurality of ultrasound output channels.

10. The device of claim 6, wherein the second ultrasound signal has a pressure amplitude less than a cavitation threshold.

11. The device of claim 10, wherein the second ultrasound output unit focuses the second ultrasound signal on the sound field, induces scattering of the second ultrasound signal from the microparticle cluster within the sound field, and generates a scattered sound field having a pressure amplitude equal to or more than the cavitation threshold from the scattering of the second ultrasound signal.

12. The device of claim 11, wherein the second ultrasound output unit uses the second ultrasound signal to induce collapse of the bubbles and the microparticle cluster generated by the scattered sound field, thereby fragmenting at least a portion of the tissue.

13. The device of claim 12, wherein the cavitation threshold is a minimum pressure at which the bubbles are generated when only the second ultrasound signal is used.

14. A method for generating bubbles based on microparticles, comprising:
outputting a first ultrasound signal to move microparticles in a blood vessel to a predetermined site; and
outputting a second ultrasound signal to generate bubbles based on the microparticles.

15. The method of claim 14, wherein the outputting of the second ultrasound signal includes outputting the second ultrasound signal toward the microparticles or surroundings of the microparticles.

16. The method of claim 15, wherein the outputting of the second ultrasound signal includes generating the bubbles based on the second ultrasound signal reflected and scattered from the microparticles or the surroundings of the microparticles.

17. The method of claim 14, wherein the outputting of the first ultrasound signal includes generating a sound field that captures the microparticles using the first ultrasound signal.

18. The method of claim 17, wherein the outputting of the second ultrasound signal includes outputting the second ultrasound signal toward the sound field and generating the bubbles based on the second ultrasound signal reflected and scattered from a microparticle cluster composed of the microparticles captured within the sound field.

19. The method of claim 17, wherein the outputting of the first ultrasound signal includes determining a size of a ring according to a pressure distribution of the sound field by adjusting a topological charge of the first ultrasound signal, and determining a size of an ultrasound focal area according to the size of the ring.

20. The method of claim 14, wherein the outputting of the first ultrasound signal includes outputting a plurality of channel signals having different phase differences through a plurality of ultrasound output channels.

21. The method of claim 18, wherein the outputting of the second ultrasound signal includes focusing the second ultrasound signal on the sound field, inducing scattering of the second ultrasound signal from the microparticle cluster within the sound field, and generating a scattered sound field having a pressure amplitude equal to or more than the cavitation threshold from the scattering of the second ultrasound signal.

22. The method of claim 21, wherein the outputting of the second ultrasound signal includes using the second ultrasound signal to induce collapse of the bubbles and the microparticle cluster generated by the scattered sound field, thereby fragmenting at least a portion of the tissue.
